(19) United States
(12) Patent Application Publication
(10) Pub. No.: US 2017/0239103 A1
(43) Pub. Date: Aug. 24, 2017

(54) ABSORBENT ARTICLE WITH ACCESSIBLE POCKET

(71) Applicant: Kimberly-Clark Worldwide, Inc., Neenah, WI (US)

(72) Inventors: Paul A. Weber, Neenah, WI (US); Sara Stabelfeldt, Appleton, WI (US); Nicole J. Barna, Appleton, WI (US); Nancy E. Dawson, Appleton, WI (US); Jeffrey A. DeBroux, Appleton, WI (US); John T. Hahn, Merrill, WI (US); Eric D. Johnson, Larsen, WI (US); Georgia L. Zehner, Larsen, WI (US)

(21) Appl. No.: 15/518,947

(22) PCT Filed: Oct. 31, 2014
(86) PCT No.: PCT/US2014/063485

§ 371 Date: Apr. 13, 2017

(51) Int. Cl. A61F 13/49; A61F 13/84; A61F 13/58

(52) U.S. Cl. CPC A61F 13/49; A61F 13/58; A61F 13/84; A61F 13/49011

(57) ABSTRACT

An absorbent article (10) can include a pocket (64) disposed in the front waist region 12. The pocket (64) can include a first side edge (70), a second side edge (72), an upper lateral edge (74), and a lower lateral edge (76). The pocket (64) can be closed with respect to the absorbent assembly (36) at least at the first side edge (70) and the second side edge (72) and can be open with respect to the absorbent assembly (36) at least at the lower lateral edge (76). The pocket (64) can be configured such that it has improved accessibility for a caregiver.

### TECHNICAL FIELD

The present invention relates to absorbent articles.

### BACKGROUND OF THE DISCLOSURE

When absorbent articles become soiled with exudates and are changed from the wearer, it is common for the lower abdomen and/or crotch region of the wearer to become soiled by urine, fecal matter, and/or other bodily discharges. Prior to replacing the soiled absorbent article and replacing it with a new, clean absorbent article, the skin of the wearer is cleansed. This cleaning of the skin can be done in a variety of ways and using a variety of different materials, but caregivers commonly use wet wipes or cloths to clean the wearer's skin. In some circumstances, caregivers may choose to use a clean portion of an inner layer of the soiled absorbent article to provide a first wipe to cleanse the wearer's skin in the lower abdomen or crotch region prior to using wet wipes, cloths, or tissues.

To perform this initial wipe, a caregiver may attempt to pinch or gather the front waist region of the absorbent article to obtain a grip on the absorbent article to use the inner layer of the absorbent article in a wiping fashion. However, pinching or gathering the front waist region of the absorbent article can reduce the effective area of the inner layer of the absorbent article that is intended to wipe the wearer's skin in the soiled area as well as create an uneven inner surface of the absorbent article that is not as conducive to wiping as the initial flat surface. Pinching or gathering the front waist region of the absorbent article in this fashion may also expose a caregiver's fingers or hand to the exudates remaining on the wearer's skin, as the gathered material in the front waist region may fold over due to pinching or gathering of the absorbent article near the front waist edge of the absorbent article where the absorbent article may have less structural integrity and/or due to the wiping motion of the caregiver employs with the absorbent article. Additionally, gripping the front waist region of the absorbent article in such a fashion may prove to be difficult altogether as the outer cover materials may have a low coefficient of friction, resulting in the gathered or pinched area of the front waist region slipping out of the caregiver's hands while trying to wipe the soiled area.

A prior document has attempted to address these issues by the introduction of a pocket in the front waist region of the absorbent article to assist with cleaning the wearer. GB 2389300A discloses various embodiments of absorbent articles including such a pocket. However, past proposed embodiments including a pocket fail to appreciate the difficulties in a caregiver using and/or accessing such a pocket.

Thus, there remains a need for an absorbent article that can provide improved functionality for the caregiver to utilize the absorbent article as a first wipe to cleanse the wearer's skin. There also remains a need for an absorbent article that includes a pocket with improved accessibility that can be employed to utilize the absorbent article in a wiping fashion.

### SUMMARY OF THE DISCLOSURE

In one embodiment, an absorbent article can include a front waist region including a front waist edge, a rear waist region including a rear waist edge, and a crotch region. The absorbent article can further include a longitudinal axis and a lateral axis defining a horizontal plane when the absorbent article is in a stretched, laid flat configuration. The absorbent article can also include an absorbent assembly including a bodyside liner, an outer cover, and an absorbent body disposed between the bodyside liner and the outer cover. The outer cover can include a body facing surface and a garment facing surface. The absorbent article can additionally include a pocket disposed in the front waist region. The pocket can include a pocket longitudinal axis, a first side edge, a second side edge opposite from the first side edge, an upper lateral edge, a lower lateral edge, and a bottom surface. The pocket can be closed with respect to the absorbent assembly at the first side edge, the second side edge, and the upper lateral edge. The pocket can be open with respect to the bottom surface at the lower lateral edge such that when the absorbent article is in the stretched, laid flat configuration, a vertical gap is provided between the lower lateral edge and the bottom surface of the pocket at the pocket longitudinal axis, the vertical gap providing access to the pocket.

In another embodiment, an absorbent article can include a front waist region including a front waist edge, a rear waist region including a rear waist edge, and a crotch region. The absorbent article can further include a longitudinal axis and a lateral axis defining a horizontal plane when the absorbent article is in a stretched, laid flat configuration. The absorbent article can also include an absorbent assembly including a bodyside liner, an outer cover, and an absorbent body disposed between the bodyside liner and the outer cover. The outer cover can include a body facing surface and a garment facing surface. The absorbent article can additionally include a pocket disposed in the front waist region. The pocket can include a pocket longitudinal axis, a first side edge, a second side edge opposite from the first side edge, an upper lateral edge, and a lower lateral edge. The pocket can be closed with respect to the absorbent assembly at the first side edge, the second side edge, and the upper lateral edge. The pocket can be open with respect to the outer cover at the lower lateral edge, wherein a portion of the outer cover forms a bottom surface of the pocket. The bottom surface of the pocket can be elastic near the lower lateral edge of the pocket such that when the absorbent article changes from the stretched, laid flat configuration to a relaxed configuration, the bottom surface of the pocket elastically retracts to provide a separation between the lower lateral edge and the bottom surface of the pocket at the longitudinal axis of the pocket, the separation providing access to the pocket.

In yet another embodiment, an absorbent article can include a front waist region including a front waist edge, a rear waist region including a rear waist edge, and a crotch region. The absorbent article can further include a longitudinal axis and a lateral axis defining a horizontal plane when the absorbent article is in a stretched, laid flat configuration. The absorbent article can also include an absorbent assembly including a bodyside liner, an outer cover, and an absorbent body disposed between the bodyside liner and the outer cover. The outer cover can include a body facing surface and a garment facing surface. The absorbent article can additionally include a pocket disposed in the front waist region. The pocket can be formed from a material. The pocket can include a first side edge, a second side edge opposite from the first side edge, an upper lateral edge, and a lower lateral edge. The pocket can be closed with respect to the absorbent assembly at least at the first side edge and the second side edge. The pocket can be open with respect to the absorbent assembly at the lower lateral edge. The pocket can include a projection in a central region of the pocket near the lower lateral edge. The projection can either be coupled to the material forming the pocket or can be integral to the material forming the pocket. The projection can provide access to the pocket.

In still yet another embodiment, an absorbent article can include a front waist region including a front waist edge, a rear waist region including a rear waist edge, and a crotch region. The absorbent article can further include a longitudinal axis and a lateral axis defining a horizontal plane when the absorbent article is in a stretched, laid flat configuration. The absorbent article can also include an absorbent assembly including a bodyside liner, an outer cover, and an absorbent body disposed between the bodyside liner and the outer cover. The outer cover can include a body facing surface and a garment facing surface. The absorbent article can additionally include a pocket disposed in the front waist region. The pocket being can be formed from a material. The pocket including a first side edge, a second side edge opposite from the first side edge, an upper lateral edge, and a lower lateral edge. The pocket can be closed with respect to the absorbent assembly at least at the first side edge and the second side edge. The pocket can be open with respect to the absorbent assembly at the lower lateral edge. The pocket can include a projection coupled to the material forming the pocket. The projection can provide access to the pocket.

### BRIEF DESCRIPTION OF DRAWINGS

A full and enabling disclosure thereof, directed to one of ordinary skill in the art, is set forth more particularly in the remainder of the specification, which makes reference to the appended figures in which:

FIG. 1 is a top plan view of an exemplary embodiment of an absorbent article including a pocket, the absorbent article being in a stretched, laid flat configuration, with the outer cover facing the viewer.

FIG. 2 is a perspective view of the absorbent article of FIG. 1 in an unfastened configuration.

FIG. 3 is a perspective view of the absorbent article of FIG. 1 in a fastened configuration.

FIG. 4A is a cross-sectional view taken along line 4-4 from FIG. 1.

FIG. 4B is a cross-sectional view taken along line 4-4 from FIG. 1, where the slack in the pocket is removed such that the vertical gap can be measured.

FIG. 5A is a cross-sectional view similar to FIG. 4, but showing an alternative embodiment of an absorbent article including a pocket, when the front fastener has not yet been coupled to the back fasteners in a fastened configuration.

FIG. 5B is another cross-sectional view of the embodiment of FIG. 5A after the front fastener has been engaged by the back fasteners in a fastened configuration, the lower lateral edge of the pocket being extended after the back fasteners have been disengaged from the front fastener.

FIG. 6 is a detailed, top plan view illustrating the front waist region of an alternative embodiment of an absorbent article including a pocket.

FIG. 7 is a cross-sectional view taken along line 7-7 from FIG. 6.

FIG. 8 is a detailed, top plan view illustrating the front waist region of an alternative embodiment of an absorbent article including a pocket having a projection that is integral to the material forming the pocket.

FIG. 9A is a detailed, top plan view illustrating the front waist region of an alternative embodiment of an absorbent article including a pocket having a projection that is coupled to the material forming the pocket.

FIG. 9B is a detailed, top plan view illustrating the front waist region of an alternative embodiment of an absorbent article including a pocket having a projection that is coupled to the material forming the pocket.

FIG. 9C is a detailed, top plan view illustrating the front waist region of an alternative embodiment of an absorbent article including a pocket having a projection that is coupled to the material forming the pocket.

FIG. 10 is a detailed, top plan view illustrating the front waist region of an alternative embodiment of an absorbent article including a pocket having a projection that is coupled to the material forming the pocket.

Repeat use of reference characters in the present specification and drawings is intended to represent the same or analogous features or elements of the disclosure.

### DETAILED DESCRIPTION OF THE DISCLOSURE

In an embodiment, the present disclosure is generally directed towards an absorbent article having a pocket disposed on the outer surface in the front waist region of the absorbent article that provides improved accessibility for a caregiver. The pocket can aid a caregiver with providing an initial cleaning of the wearer after the article is soiled by the wearer and prior to changing the absorbent article. Each example is provided by way of explanation and is not meant as a limitation. For example, features illustrated or described as part of one embodiment or figure can be used on another embodiment or figure to yield yet another embodiment. It is intended that the present disclosure include such modifications and variations.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, training pants, youth pants, swim pants, feminine hygiene products, including, but not limited to, menstrual pads, incontinence products, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, training pant, youth pant, incontinent product, or other absorbent article about the wearer of one of these absorbent articles. A user and a wearer can be one and the same person.

Absorbent Article:

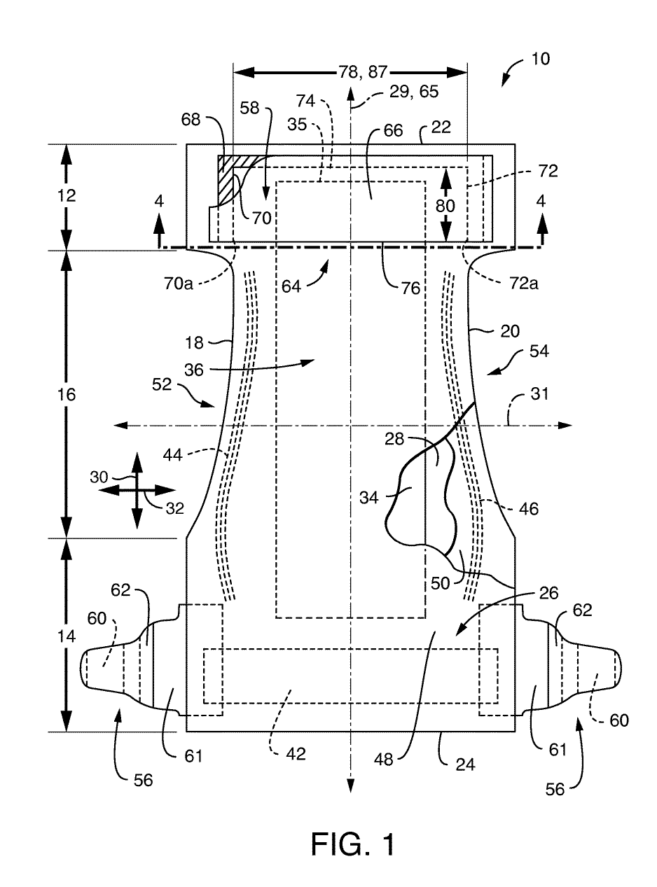

Referring to FIGS. 1-3, a non-limiting illustration of an absorbent article 10, for example, a diaper, is illustrated. Other embodiments of the absorbent article could include training pants, youth pants, adult incontinence garments, and feminine hygiene articles. While the embodiments and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross direction manufacturing of a product, without departing from the spirit and scope of the disclosure.

The absorbent article 10 illustrated in FIG. 1 includes a front waist region 12, a rear waist region 14, and a crotch region 16 disposed between the front waist region 12 and the rear waist region 14 and interconnecting the front and rear waist regions, 12, 14, respectively. The front waist region 12 can be referred to as the front end region, the rear waist region 14 can be referred to as the rear end region, and the crotch region 16 can be referred to as the intermediate region. The absorbent article 10 has a pair of longitudinal side edges, 18, 20, and a pair of opposite waist edges, respectively designated front waist edge 22 and rear waist edge 24. The front waist region 12 can be contiguous with the front waist edge 22 and the rear waist region 14 can be contiguous with the rear waist edge 24. The longitudinal side edges 18, 20 can extend from the front waist edge 22 to the rear waist edge 24.

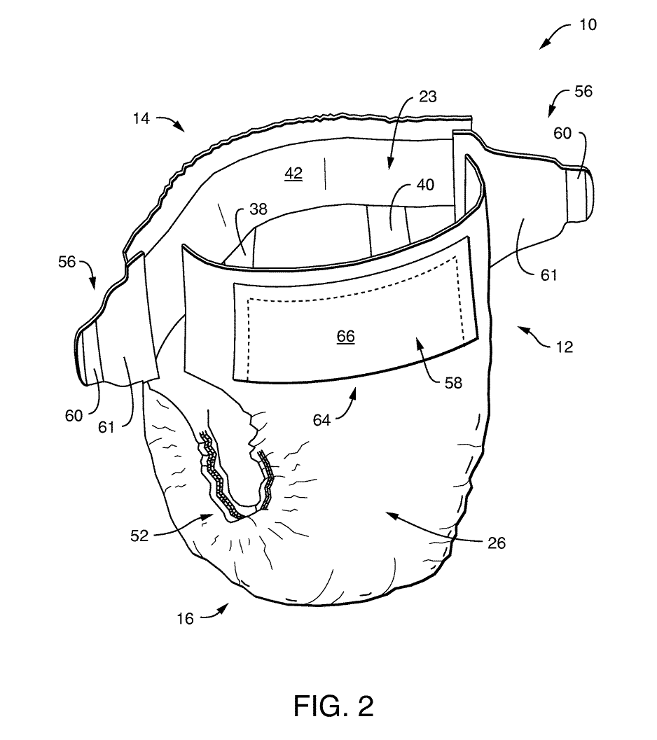

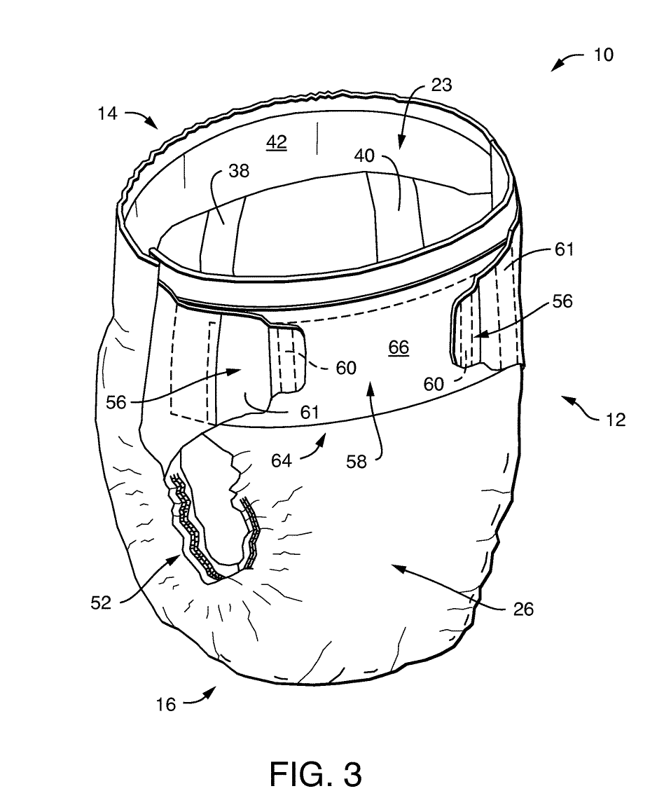

The front waist region 12 can include the portion of the absorbent article 10 that, when worn (such as in a fastened configuration as depicted in FIG. 3,) is positioned at least in part on the front of the wearer. The rear waist region 14 can include the portion of the absorbent article 10 that, when worn (such as in a fastened configuration as depicted in FIG. 3), is positioned at least in part on the back of the wearer. The crotch region 16 of the absorbent article 10 can include the portion of the absorbent article 10, that, when worn, is positioned between the legs of the wearer and can partially cover the lower torso of the wearer. The waist edges, 22 and 24, of the absorbent article 10 are configured to encircle the waist of the wearer and together define a central waist opening 23 for the waist of the wearer, as shown in FIGS. 2 and 3. Portions of the longitudinal side edges, 18 and 20, in the crotch region 16 can generally define leg openings for the legs of the wearer when the absorbent article 10 is worn.

The absorbent article 10 can include an outer cover 26 and a bodyside liner 28, the bodyside liner 28 being depicted in the cut-away portion of FIG. 1. In an embodiment, the bodyside liner 28 can be bonded to the outer cover 26 in a superposed relation by any suitable means such as, but not limited to, adhesives, ultrasonic bonds, thermal bonds, pressure bonds, or other conventional techniques. The outer cover 26 can define a length in a longitudinal direction 30, and a width in the lateral direction 32, which, in the illustrated embodiment, can coincide with the length and width of the absorbent article 10. As illustrated in FIG. 1, the absorbent article 10 can have a longitudinal axis 29 extending in the longitudinal direction 30 and a lateral axis 31 extending in the lateral direction 32. The longitudinal axis 29 and the lateral axis 31 can define a horizontal plane.

An absorbent body 34 can be disposed between the outer cover 26 and the bodyside liner 28. In an embodiment, the absorbent body 34 can have a length and width that are the same as or less than the length and width of the absorbent article 10. The bodyside liner 28, the outer cover 26, and the absorbent body 34 can form part of an absorbent assembly 36. The absorbent assembly 36 can also include other components not shown herein, such as a fluid transfer layer and a fluid acquisition layer, as are known in the art.

The absorbent article 10 can be configured to contain and/or absorb liquid, solid, and semi-solid body exudates discharged from the wearer. For example, containment flaps 38, 40 (shown in FIGS. 2 and 3), which are known in the art, can be configured to provide a barrier to the lateral flow of body exudates. To further enhance containment and/or absorption of body exudates, in some embodiments the absorbent article 10 can suitably include a waist elastic member, such as a rear waist elastic member 42. In some embodiments, the absorbent article 10 can include a front waist elastic member, although one is not depicted in the figures herein. The absorbent article 10 can further include leg elastic members, 44 and 46, as depicted in FIGS. 1-3 and as are known to those skilled in the art. The rear waist elastic member 42 can be attached to the outer cover 26 and/or the bodyside liner 28 along the rear waist edge 24 and can extend over part or all of the rear waist edge 24. In an embodiment shown in FIGS. 2 and 3, the rear waist elastic member 42 is attached to the bodyside liner 28. The leg elastic members, 44 and 46, can be attached to the outer cover 26 and/or the bodyside liner 28 along the opposite longitudinal side edges, 18 and 20, and positioned in the crotch region 16 of the absorbent article 10. The leg elastic members, 44 and 46, can be curved as shown in FIG. 1, or can be parallel to the longitudinal axis 29 as is known in the art.

The absorbent article 10 can also include a fastening system that can be used to put the article 10 in a fastened configuration, such as shown in FIG. 3. In some embodiments, the fastening system can include a pair of back fasteners 56 and at least one front fastener 58. The back fasteners 56 can each include a fastening component 60 that can engage the front fastener 58.

Additional details regarding each of these elements of the absorbent article 10 described herein can be found below and with reference to the FIGS. 1 through 10.

Outer Cover:

The outer cover 26 and/or portions thereof can be breathable and/or liquid impermeable. The outer cover 26 and/or portions thereof can be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 26 can be constructed of a microporous polymeric film, such as polyethylene or polypropylene.

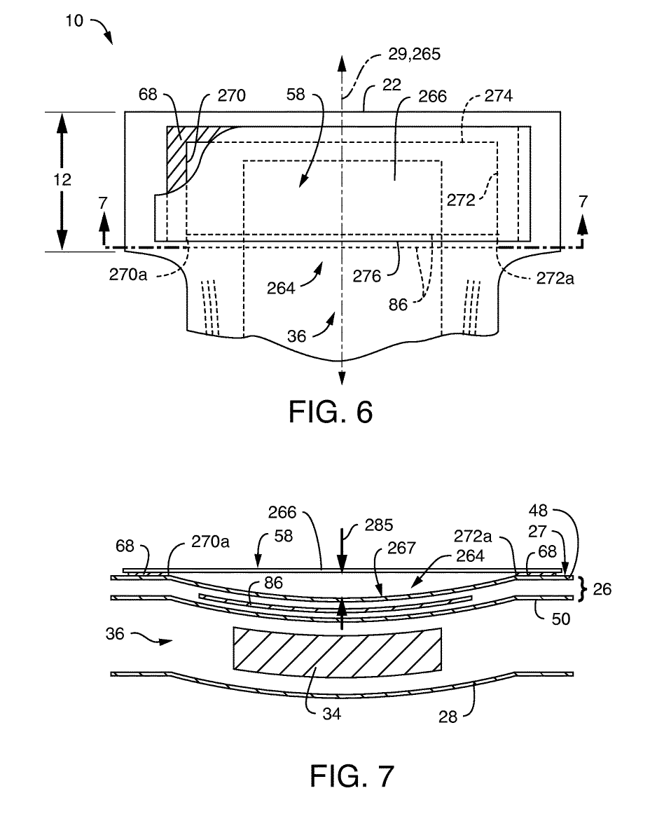

In an embodiment, the outer cover 26 can be a single layer of a liquid impermeable material, such as a polymeric film. In an embodiment, the outer cover 26 can be suitably stretchable, and more suitably elastic, in at least the lateral direction 32 of the absorbent article 10. In an embodiment, the outer cover 26 can be stretchable, and more suitably elastic, in both the lateral 32 and the longitudinal 30 directions. In an embodiment, the outer cover 26 can be a multi-layered laminate in which at least one of the layers is liquid impermeable. In an embodiment, such as that illustrated in FIGS. 1 and 6, the outer cover 26 can be a two layer construction, including an outer layer 48 material and an inner layer 50 material (labeled in FIGS. 1 and 7) which can be bonded together such as by a laminate adhesive. Suitable laminate adhesives can be applied continuously or intermittently as beads, a spray, parallel swirls, or the like, but it is to be understood that the inner layer 50 can be bonded to the outer layer 48 by other bonding methods, including, but not limited to, ultrasonic bonds, thermal bonds, pressure bonds, or the like.

The outer layer 48 of the outer cover 26 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer 48 of an outer cover 26 can be a 20 gsm spunbond polypropylene non-woven web. The outer layer 48 may also be constructed of the same materials from which the bodyside liner 28 can be constructed as described herein.

The liquid impermeable inner layer 50 of the outer cover 26 (or the liquid impermeable outer cover 26 where the outer cover 26 is of a single-layer construction) can be either vapor permeable (i.e., “breathable”) or vapor impermeable. The liquid impermeable inner layer 50 (or the liquid impermeable outer cover 26 where the outer cover 26 is of a single-layer construction) can be manufactured from a thin plastic film. The liquid impermeable inner layer 50 (or the liquid impermeable outer cover 26 where the outer cover 26 is of a single-layer construction) can inhibit liquid body exudates from leaking out of the absorbent article 10 and wetting articles, such as bed sheets and clothing, as well as the wearer and caregiver.

Where the outer cover 26 is of a single layer construction, it can be embossed and/or matte finished to provide a more cloth-like texture or appearance. The outer cover 26 can permit vapors to escape from the absorbent article 10 while preventing liquids from passing through. A suitable liquid impermeable, vapor permeable material can be composed of a microporous polymer film or a non-woven material which has been coated or otherwise treated to impart a desired level of liquid impermeability.

As shown in FIGS. 1-4B, the absorbent article 10 can include a pocket 64. The pocket 64 can be disposed in the front waist region 12 of the absorbent article 10. In some embodiments, the pocket 64 can be disposed in the front waist region 12 and extend into the crotch region 16 of the absorbent article 10. The pocket 64 can include a pocket longitudinal axis 65, as shown in FIG. 1. Preferably, the pocket longitudinal axis 65 substantially aligns with the longitudinal axis 29 of the absorbent article 10. In some embodiments, the pocket 64 can be formed from a material 66 coupled to the outer cover 26. The material 66 can be coupled to the outer cover 26 by any suitable method known in the art, such as by adhesive 68, as shown in the embodiment of the absorbent article 10 in FIGS. 1-4. In other embodiments, the pocket 64 can be formed from one or more components of the absorbent assembly 36, such as the outer cover 26.

The pocket 64 can include a first side edge 70 and a second side edge 72. The second side edge 72 can be opposite from the first side edge 70. The pocket 64 can also include an upper lateral edge 74 and a lower lateral edge 76. The pocket 64 can include a bottom surface 67. The pocket 64 can be closed with respect to the absorbent assembly 36 at least at the first side edge 70 and the second side edge 72. In some embodiments, the pocket 64 can be closed with respect to the absorbent assembly 36 at the upper lateral edge 74 as well. As shown in the embodiment depicted in FIGS. 1, 6, and 8-10, the pocket 64, 264, 364, 464 is closed with respect to the absorbent assembly 36 in this manner due to the selective location of the adhesive 68. The adhesive 68 can bond the material 66 forming the pocket 64 to the garment facing surface 27 of the outer cover 26. The pocket 64 can be open with respect to the absorbent assembly 36 at the lower lateral edge 76. The open nature of the pocket 64 at the lower lateral edge 76 allows a caregiver's hand to enter the pocket 64 to assist with an initial wiping of the skin of the wearer after the article 10 becomes soiled with exudates prior to disposing of the soiled absorbent article 10 and cleansing the wearer's skin, as will be discussed in further detail below. In some embodiments, the pocket 64 can be open with respect to absorbent assembly 36 at the upper lateral edge 74 as well as at the lower lateral edge 76.

As illustrated in FIG. 1, the pocket 64 can include a width 78 and a length 80. The length 80 can be measured in the longitudinal direction 30 from the upper lateral edge 74 to the lower lateral edge 76. In preferred embodiments, the length 80 of the pocket 64 can be between about 1.25 inches and about 3.75 inches. The width 78 can be measured in the lateral direction 32 from the first side edge 70 to the second side edge 72. In preferred embodiments, the width 78 of the pocket 64 can be between about 3.00 inches and about 6.00 inches.

The pocket 64 can be configured to provide improved accessibility for a caregiver to place their hand or a portion of their hand in the pocket 64. This can be achieved in several different configurations.

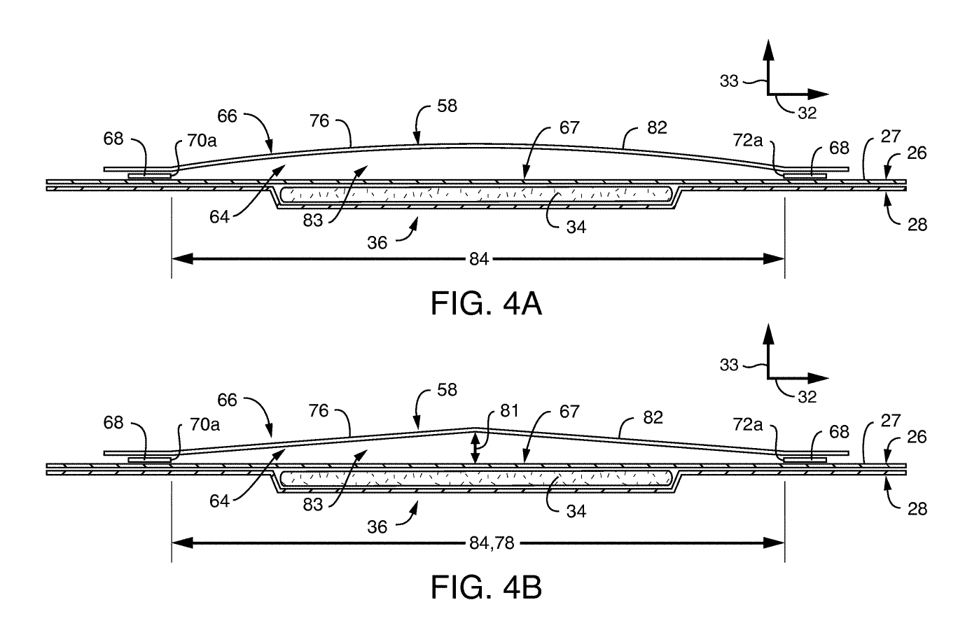

For example, with reference to FIGS. 4A and 4B, the pocket 64 can be open with respect to the bottom surface 67 at the lower lateral edge 76 of the pocket 64 such that when the absorbent article 10 is in the stretched, laid flat configuration, a vertical gap 81 can be provided between the lower lateral edge 76 and the bottom surface 67 of the pocket 64 at the pocket longitudinal axis 65 (labeled in FIG. 1). For purposes herein, the vertical gap 81 is to be measured as the distance between the lower lateral edge 76 of the pocket 64 and the bottom surface 67 of the pocket 64 measured in the vertical direction 33 when the slack is removed from the material 66 forming the pocket 64, such as illustrated in FIG. 4B, when the absorbent article is in the stretched, laid flat configuration. Of note, removing the slack from the material 66 forming the pocket 64 in order to measure the vertical gap 81 does not include elastically extending the material 66 if the material 66 is elastic or extending the material 66 in such a manner that deforms the material 66 if the material 66 is not elastic, but is extendable. The vertical direction 33 is perpendicular to the horizontal plane defined by the longitudinal axis 29 and the lateral axis 31 when the absorbent article 10 is in a stretched, laid flat configuration. The vertical gap 81 can be measured with a ruler when the absorbent article 10 is placed in the stretched, laid flat configuration and the slack is removed from the material 66 forming the pocket 64. Thus, any values relating to the vertical gap 81 as discussed herein relate to this measurement method for the vertical gap 81.

As shown in FIGS. 4A and 4B, the vertical gap 81 can provide improved accessibility to the pocket 64, even when the absorbent article 10 is in the stretched, laid flat configuration. The configuration of the vertical gap 81 and the width 78 of the pocket 64 at the lower lateral edge 76 can define an open area 83 for a caregiver's hand or finger(s) to enter the pocket 64. The size of the vertical gap 81 can vary between different embodiments. In some embodiments, the vertical gap 81 can be from about 0.0625 inches to about 3.00 inches, more preferably from about 0.125 inches to about 2.00 inches, and more preferably from about 0.25 inches to about 1.00 inches. Of course, it is contemplated that the vertical gap 81 can be outside of these preferable ranges. In some circumstances, the size of the vertical gap 81 can be configured to be larger if the width 78 of the pocket 64 is configured to be smaller as compared to other embodiments in an effort to achieve similar open area 83 for the caregiver's hand or finger(s) to enter the pocket 64. In some embodiments, a ratio of the width 78 of the pocket 64 to the vertical gap 81 can be less than about 100:1, less than about 90:1, less than about 80:1, less than about 70:1, less than about 60:1, less than about 50:1, less than about 40:1, less than about 30:1, less than about 25:1, less than about 20:1, less than about 19:1, less than about 18:1, less than about 17:1, less than about 16:1, less than about 15:1, less than about 14:1, less than about 13:1, less than about 12:1, less than about 11:1, less than about 10:1, less than about 9:1, less than about 8:1, less than about 7:1, less than about 6:1, less than about 5:1, less than about 4:1, less than about 3:1, less than about 2:1, or about 1:1.

In some embodiments, the vertical gap 81 can be created for the pocket 64 by having the length 82 along the lower lateral edge 76 of the pocket 64 from the first side edge 70 to the second side edge 72 be greater than the length 84 along the garment facing surface 27 of the outer cover 26 from the first side edge 70 to the second side edge 72, as illustrated in FIGS. 4A and 4B. The length 82 measured along the lower lateral edge 76 of the pocket 64 follows the actual path of the lower lateral edge 76 in the longitudinal, lateral, and vertical directions, 30, 32, 33, respectively. The length 84 along the garment facing surface 27 of the outer cover 26 is measured along a projected path of the lower lateral edge 76 in the horizontal plane defined by the longitudinal axis 29 and the lateral axis 31. Thus, the length 84 can be, but is not necessarily measured along a straight line in the lateral direction 32 between the distal end 70a of the first side edge 70 of the pocket 64 and the distal end 72a of the second side edge 72 of the pocket 64. For example, in the embodiment shown in FIGS. 1-4B where the lower lateral edge 76 of the pocket 64 is configured to be a linear segment in the lateral direction 32, the length 84 along the outer cover 26 will follow a projected path of the lower lateral edge 76 that will be linear between the distal end 70a of the first side edge 70 of the pocket 64 and the distal end 72a of the second side edge 72 of the pocket 64. However, if the lower lateral edge 76 was arcuate in nature in the lateral direction 32, or some other non-linear shape, then the length 84 along the outer cover 26 will follow a projected path of such arcuate shape (or other non-linear shape) of the lower lateral edge 76 in the horizontal plane defined by the longitudinal axis 29 and the lateral axis 31, and therefore, the length 84 along the outer cover 26 will not be measured in a linear fashion between the distal end 70a of the first side edge 70 of the pocket 64 and the distal end 72a of the second side edge 72 of the pocket 64.

Where the lower lateral edge 76 of the pocket 64 forms a linear segment, such as in the embodiment depicted in FIGS. 1-4B, the length 82 of the lower lateral edge 82 can be measured with a ruler. This can be accomplished by measuring the length of the two angled sections of the lower lateral edge 76 when the slack is removed from the pocket 64 and the absorbent article 10 is in the stretched, laid flat configuration, such as illustrated in FIG. 4B, and adding the length of the two angled sections to provide the length 82 of the lower lateral edge 76. Alternatively, the length 82 of the lower lateral edge 76 can be measured by removing the bond of the upper lateral edge 74 and one of either the first side edge 70 or the second side edge 72 of the pocket 64 such that the material 66 forming the pocket 64 can be laid flat, and the length 82 of the lower lateral edge 76 can thus be measured in the horizontal plane defined by the longitudinal axis 29 and the lateral axis 31. If the lower lateral edge 76 is not a linear segment, the length 82 of the lower lateral edge 76 can be calculated using appropriate mathematical formulas to model the shape of the lower lateral edge 76, as are known by those of ordinary skill in the art. It is to be noted that all measurements for the vertical gap 81, the length 82 of the lower lateral edge 76, and the length 84 along the garment facing surface 27 of the outer cover 26 from the first side edge 70 to the second side edge 72 of the pocket 64 are to be measured when the product is in the stretched, laid flat configuration and when the absorbent article has not yet been used.

It is to be noted that the length 87 (labeled in FIG. 1) of the upper lateral edge 74 as measured in the lateral direction 32 can be equal to or different from the length 82 of the lower lateral edge 76. For example, if the upper lateral edge 74 is closed with respect to the absorbent assembly 36, the length 87 of the upper lateral edge 74 can be less than the length 82 of the lower lateral edge 76 when the absorbent article is in the stretched, laid flat configuration.

As illustrated in the cross-sectional view in FIG. 4A where the material 66 forming the pocket 64 has some slack and in the cross-sectional view in FIG. 4B where the vertical gap 81 can be measured, by having the length 82 be greater than length 84, the pocket 64 provides easier access for a caregiver to enter their hand, or a portion of their hand, into the pocket 64 due to the open area 83 between the lower lateral edge 76 of the pocket 64 and the garment facing surface 27 of the outer cover 64 forming the bottom surface 67 of the pocket 64. This can be especially beneficial from the standpoint that a caregiver may be using one of their hands to hold a portion of the wearer's body or the wearer's clothing at the point in time the caregiver wants to employ the pocket 64, thus leaving only one hand to enter and use the pocket 64 for wiping the wearer's skin with a clean surface on the absorbent article 10 prior to changing the absorbent article 10. Easier accessibility into the pocket 64 allows easier operation of the pocket 64 for the caregiver.

In one embodiment, the vertical gap 81 in the pocket 64 can be created by gathering the material 66 that forms the pocket 64 prior to attaching the material 66 to the absorbent assembly 36. For example, the material 66 that forms the pocket 64 can be gathered in the lateral direction 32 prior to attaching the material 66 to the outer cover 26 of the absorbent article 10 with adhesive 68. In other words, the material 66 forming the pocket 64 is not extended to lay flat against the outer cover 26, or not extended to a stretch to stop configuration, before it is attached to the outer cover 26 of the absorbent article 10. In such a configuration, the length 82 along the lower lateral edge 76 of the pocket 64 from the first side edge 70 to the second side edge 72 can be made to be greater than a length 84 along the garment facing surface 27 of the outer cover 26 from the first side edge 70 to the second side edge 72. FIGS. 4A and 4B illustrate an embodiment of the material 66 forming the pocket 64 being gathered in the lateral direction 32 to achieve the length 82 being greater than the length 84. In such a configuration, the vertical gap 81 can be present in the absorbent article 10 in the stretched, laid flat configuration before the fastening system is engaged and disengaged for a first time.

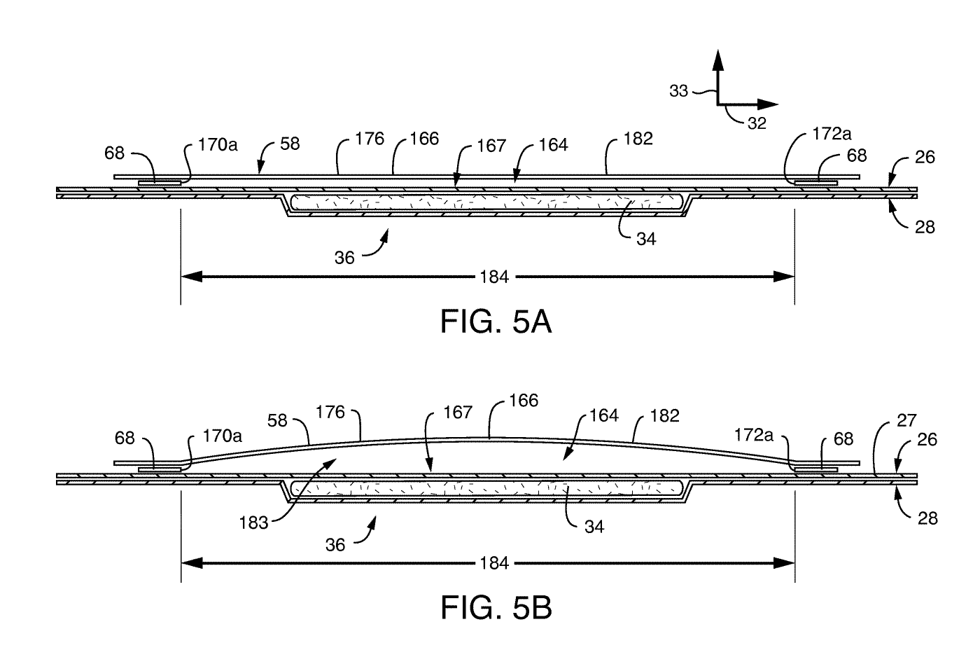

Referring now to FIGS. 5A and 5B, another embodiment of a pocket 164 is shown where the fastening system can provide access to the pocket 164 of the absorbent article 10. As illustrated in FIGS. 1-3, the absorbent article 10 can include a fastening system. The fastening system can include a pair of back fasteners 56 in the rear waist region 14 and at least one front fastener 58 in the front waist region 12. The back fasteners 56 can each include a fastening component 60. The fastening component 60, such as a hook material, can be configured to engage the front fastening component 58, which can be a loop material, such as shown in FIG. 3 in the fastened configuration. As shown in FIGS. 1-5B, the material 66, 166 forming the pocket 64, 164 can be the same material that forms the front fastening component 58. FIG. 5A illustrates a cross-sectional view of the pocket 164 prior to the back fasteners 56 engaging the front fastener 58 to place the absorbent article in a fastened configuration for the first time. In such a configuration, there is no vertical gap provided between the lower lateral edge 76 and the bottom surface 67 of the pocket 164, and thus, substantially no open area 83 for a caregiver to insert their hand or finger(s) into the pocket 164 when the absorbent article 10 is in the stretched, laid flat configuration. In such a configuration, the length 182 along the lower lateral edge 176 of the pocket 164 from the first side edge 170 to the second side edge 172 can be substantially equal to the length 184 along the garment facing surface 27 of the outer cover 26 from the first side edge 170 to the second side edge 172.

After the back fasteners 56 engage the front fastener 58 in a fastened configuration, and then the back fasteners 56 are disengaged from the front fastener 58 to an unfastened configuration for the first time, an open area 183 can be created in the pocket 164 near the lower lateral edge 176, as depicted in FIG. 5B. The open area 183 can provide for a vertical gap that can be measured as discussed above with respect to FIG. 4B. In such a configuration, the length 182 can increase to be greater than the length 184 after the fastening system has been engaged in a fastened configuration and then disengaged from the fastened configuration to an unfastened configuration, as illustrated in FIG. 5B. Such a configuration can be achieved by employing an extensible, but not elastic, material to be the material 166 forming the pocket 164. Additionally and/or alternatively, the material 166 forming the pocket 164 can be in a pleated configuration to provide such a result. In such embodiments, after the back fasteners 56 are disengaged from the front fastener 58 the material 166 forming the pocket 164 can extend in width in the lateral direction 32, such that a vertical gap is provided in the pocket 164 to create an open area 183, and the length 182 along the lower lateral edge 176 of the pocket 164 from the first side edge 170 to the second side edge 172 can be greater than the length 184 along the garment facing surface 27 of the outer cover 26 from the first side edge 170 to the second side edge 172, similar to that as discussed above with respect to FIGS. 1-4B. As mentioned above, having a vertical gap at the lower lateral edge 176 of the pocket 164 provides for easier access to the pocket 164 for the caregiver.

Embodiments such as those illustrated in FIGS. 5A and 5B where no vertical gap is present between the lower lateral edge 176 and the bottom surface 167 of the pocket 164 prior to the fastening system being disengaged for the first time can provide additional benefits. For example, in such a configuration, the pocket 164 becomes accessible for the caregiver only at the time it is desired to be used by the caregiver to assist in wiping the wearer's skin. In other words, the pocket 164, and more specifically, the lower lateral edge 176, can be adjacent to the garment facing surface 27 of the outer cover 26 prior to and while the absorbent article 10 is in the fastened configuration such that no vertical gap is present, but then the pocket 164 becomes more accessible upon the disengagement of the fasteners 56, 58 when the absorbent article 10 is in the unfastened configuration such that a vertical gap and open area 183 are provided.

Thus, the embodiments of FIGS. 1-5B provide different examples of where a vertical gap 81 can be provided in the pocket 64, 164 to provide easier accessibility to the pocket 64, 164 when the absorbent article 10 is in the stretched, laid flat configuration. Such configurations can provide for the length 82, 182 along the lower lateral edge 76, 176 of the pocket 64, 164 from the first side edge 70, 170 to the second side edge 72, 172 to be greater than the length 84, 184 along the garment facing surface 27 of the outer cover 26 from the first side edge 70, 170 to the second side edge 72, 172. As discussed above with respect to FIGS. 1-4B, the vertical gap 81 can be provided prior to the fastening system being engaged for the first time. In such configurations, the length 82 can be greater than the length 84 prior to the fastening system being engaged and disengaged for a first time. Additionally or alternatively, the vertical gap can be provided after the fastening system is disengaged from the fastened configuration to an unfastened configuration for the first time, such as illustrated in FIGS. 5A and 5B. In a sample configuration, the length 182 can become greater than the length 184 after the fastening system is disengaged for a first time. Of course, it is contemplated that such a configuration and/or material selection for the material 166 forming the pocket 164 as discussed in FIGS. 5A and 5B can be used in addition to the configuration as illustrated in FIGS. 1-4B of the gathered material 66 to provide further accessibility to the pocket 64, or as an alternative to such a configuration as illustrated in FIGS. 1-4B. Where the configuration and/or material selection for the material 166 forming the pocket 164 is in addition to the configuration as illustrated in FIGS. 1-4B where a vertical gap 81 is present prior to the fastening system being disengaged for the first time, the vertical gap 81 can increase after the fastening system is disengaged for the first time.

Another embodiment of an absorbent article 10 with a pocket 264 having increased accessibility is shown in FIGS. 6 and 7. The pocket 264 can be formed from a material 266. The material 266 can be extensible and non-elastic, elastic, or non-extendable. The pocket 264 can configured such that a portion of the outer cover 26 forms a bottom surface 267 of the pocket 264. For example, the garment facing surface 27 of the outer cover 26 can form a bottom surface 267 of the pocket 264. The bottom surface 267 of the pocket 264 can be elastic near the lower lateral edge 276 of the pocket 264 such that when the absorbent article 10 moves from a stretched, laid flat configuration to a relaxed configuration (as shown in the exploded, cross-sectional view in FIG. 7), the bottom surface 267 of the pocket 264 can elastically retract to provide a separation 285 between the lower lateral edge and the bottom surface of the pocket 264 at the longitudinal axis 265 of the pocket 264 (labeled in FIG. 6). The separation 285 provides access to the pocket 264 when the absorbent article 10 is in the relaxed configuration, as illustrated in FIG. 7, and the bottom surface 267 of the pocket 264 elastically retracts.

The elastic nature of the bottom surface 267 of the pocket 264 can be provided in a variety of ways. For example, an elastic material 86 can be coupled to the portion of the outer cover 26 that forms the bottom surface 267 of the pocket 264 near the lower lateral edge 276 of the pocket 264. As illustrated in FIGS. 6 and 7, the outer cover 26 can include an outer layer 48 and an inner layer 50. In one embodiment, the elastic material 86 can be disposed between the outer layer 48 and the inner layer 50 of the outer cover 26 near the lower lateral edge 276 of the pocket 264. As illustrated in FIGS. 6 and 7, the elastic material 86 can be one or more strands of material having elastic properties. Suitable elastic materials 86 can include sheets, strands or ribbons of natural rubber, synthetic rubber, or thermoplastic elastomeric materials. The elastic material 86 can be stretched and secured to the outer cover 26, or secured to the outer cover 26 and then elasticized or shrunk, for example, with the application of heat, such that the elastic retractive forces are imparted to the outer cover 26. It is also contemplated that the elastic nature in this region of the outer cover 26 can be provided by the elastic properties of the outer cover 26 itself. The entire outer cover 26 can be comprised of an elastic material, or a portion of the outer cover 26 near the lower lateral edge 276 of the pocket 264 can exhibit elastic properties. Although not shown, it is also contemplated that the pocket 264 could be formed by a slit in the outer layer 48 of the outer cover 26 such that the bottom surface 267 of the pocket 264 is formed by a portion of the inner layer 50 of the outer cover 26. In such an alternative configuration, the elastic material 86 can be preferably disposed between the inner layer 50 of the outer cover 26 and the bodyside liner 28.

By imparting retractive forces on the outer cover 26 near at least the lower lateral edge 276 of the pocket 264 can provide benefits, similar to those discussed above with respect to pockets 64, 164 in FIGS. 1-5B. For example, the pocket 264 can provide easier access for a caregiver to use the pocket 264. Notably, the pocket 264 can provide the benefit of remaining adjacent the garment facing surface 27 of the outer cover 26 while the absorbent article 10 is in a fastened configuration, but then the pocket 264 can provide more accessibility as the fastening system is disengaged and the article 10 moves to the relaxed configuration such that the bottom surface 267 of the pocket 264 can elastically retract and open the pocket 264 for the caregiver providing improved accessibility. It is to be noted that the elastic nature of the bottom surface 267 of the pocket 264 can be used in addition to or as an alternative to the configurations noted above with respect to pocket 164 in FIGS. 5A and 5B, and/or pocket 64 in FIGS. 1-4B. For example, the configuration of FIGS. 6 and 7 could be combined with a configuration of a pocket that has a vertical gap 81 in the stretched, laid flat configuration, such as the pocket 64 illustrated in FIGS. 1-4B, such that a vertical gap is present in the pocket 264 in the stretched, laid flat configuration to provide access to the pocket 264, and the separation 285 is created by the elastic retraction of the bottom surface 267 of the pocket 264.

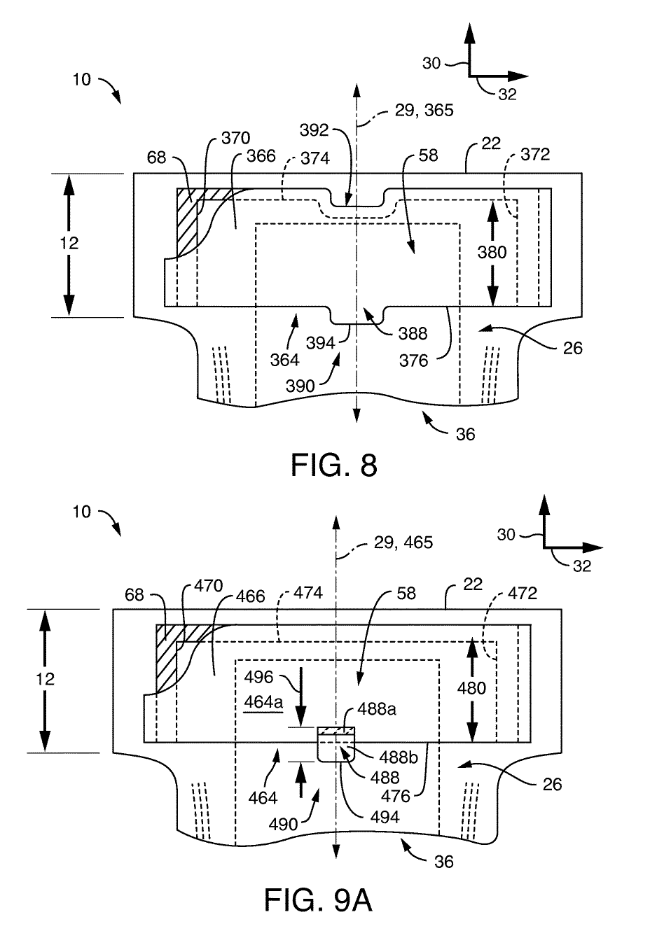

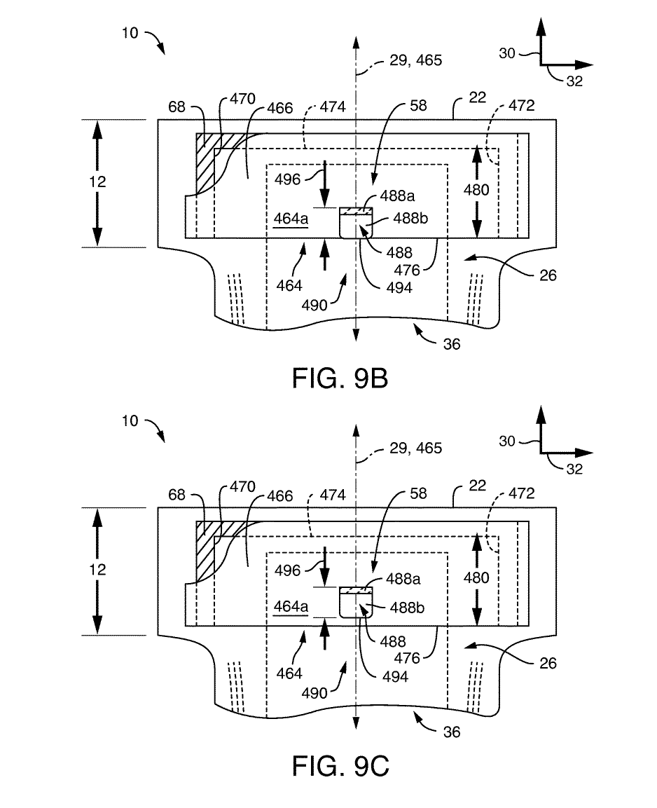

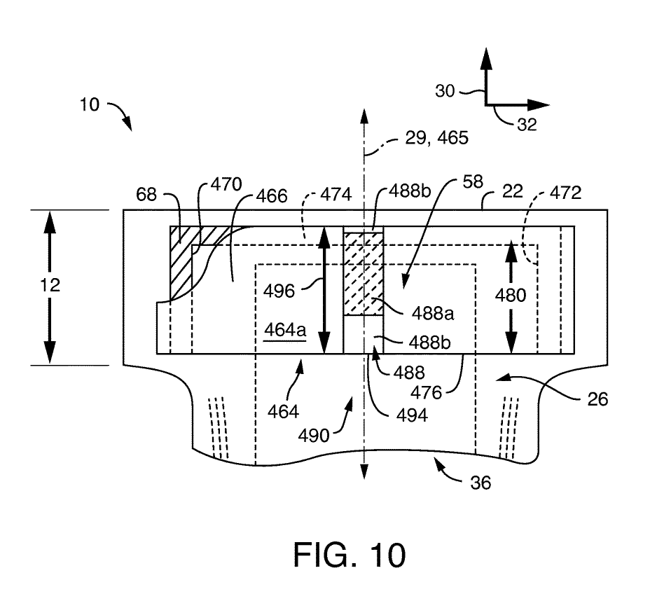

Referring now to FIGS. 8-10, other embodiments of an absorbent article 10 including a pocket 364, 464 with improved accessibility are shown. In FIGS. 8-10, the pockets 364, 464 can include a projection 388, 488 that can provide access to the pockets 364, 464. The projection 388 of FIG. 8 and the projection 488 of FIGS. 9A-10 can be in a central region 390, 490 of the pockets 364, 464 near the lower lateral edge 376, 476. The central region 390, 490 can be equal to about 50% of the lateral width of the pockets 364, 464 centered around the longitudinal axis 365, 465 of the pockets 364, 464. As shown in FIG. 8, the projection 388 can be integral with the material 366 providing the pocket 364. However, FIGS. 9A-10 provide an embodiment where the projection 488 can be coupled to the material 466 providing the pocket 464.

As illustrated in FIG. 8, the projection 388 can include a bottom edge 394 that forms a portion of the lower lateral edge 376 of the pocket 364. The bottom edge 394 of the projection 388 can extend closer to the lateral axis 31 (not shown in FIG. 8) of the absorbent article 10 than is the portion of the lower lateral edge 376 that does not include the projection 388. As also illustrated in FIG. 8, the material 366 forming the pocket 364 can include a notch 392 near the upper lateral edge 374. The notch 392 can have a shape that nests with the projection 388 that is formed integral with the material 366 forming the pocket 364. The projection 388 and the notch 392 can be nested due to a die-cut manufacturing method of cutting the material 366 forming the pocket 364, such that a projection 388 in one pocket 364 will be nested with the notch 392 of an adjacent pocket 364 as the material 366 is being cut by a die cutter (not shown). Of course, the notch 392 near the upper lateral edge 374 is an optional feature in the pocket 364.

As illustrated in FIGS. 9A-10, the pocket 464 can alternatively include a projection 488 that is coupled to the material 466 forming the pocket 464. The projection 488 can included at least one bonded portion 488a and at least one non-bonded portion 488b. The bonded portion(s) 488a can attach the projection 488 to the material 466 forming the pocket 464 and the non-bonded portion(s) 488b can be not attached to the material 466 forming the pocket 464. The projection 488 can be coupled to the material 466 forming the pocket 464 at the bonded portion(s) 488a via adhesive, pressure bonding, ultrasonic bonding, thermal bonding, stitching, or any other suitable means known by those of ordinary skill in the art. As illustrated in FIGS. 9A-9C, the bonded portion(s) 488a can have less area than the area of the non-bonded portion(s) 488b of the projection 488.

The projection 488 can be coupled to the material 466 forming the pocket 464 in a variety of configurations. For example, as illustrated in FIGS. 9B, 9C, and 10, at least one of the non-bonded portions 488b of the projection 488 can be disposed between the upper lateral edge 474 and the lower lateral edge 476 of the pocket 464. As illustrated in FIG. 9A, at least a portion of a non-bonded portion 488b can extend beyond the lower lateral edge 476 of the pocket 464 such that at least a portion of the non-bonded portion 488b is disposed between the lower lateral edge 476 of the pocket 464 and the lateral axis 31 (not shown in FIG. 9A) of the absorbent article 10. Stated differently, the bottom edge 494 of the projection 488 can extend closer to the lateral axis 31 (not shown in FIG. 9A) of the absorbent article 10 than does the lower lateral edge 476 of the pocket 464. As illustrated in FIGS. 9B and 10, the projection 488 can be coupled to the material 466 forming the pocket 464 such that the bottom edge 494 of the projection 488 is substantially aligned with the lower lateral edge 476 of the pocket 464. As depicted in FIGS. 9A-10, the projection 488 is preferably coupled to the garment facing surface 464a of the pocket 464, however, in configurations where the projection 488 extends beyond the lower lateral edge 476 of the pocket 464 (such as in FIG. 9A), it is contemplated that projection 488 could be coupled to the body facing surface of the pocket 464.

The projection 488 can be configured to be of various sizes and shapes. For example, in FIGS. 9A-9C the projection 488 can have a length 496 in the longitudinal direction 30 that is less than the length 480 of the pocket 464. However, as illustrated in FIG. 10, the projection 488 can be configured to be equal to or greater in length 496 than the length 480 of the pocket 464. As depicted in FIG. 10, the projection 488 can be configured to extend from the upper lateral 474 to at least the lower lateral edge 476. The projection 488 can also extend above the upper lateral edge 474 and/or below the lower lateral edge 476. While the projections 488 depicted herein are generally rectangular in shape, it is contemplated that the projection 488 may be configured in other suitable shapes, including, but not limited to circles, semi-circles, triangles, arcs, ellipses, and other suitable shapes.

The projections 388, 488 as depicted in FIGS. 8-10 provide for improved accessibility to the pocket 364, 464 of the absorbent article 10 by providing the caregiver with a gripping point to lift the pocket 364, 464 away from the absorbent assembly 36 such that the caregiver's hand or a portion of their hand can more easily enter the pocket 364, 464. With respect to the projections 488 depicted in FIGS. 9A-10, the non-bonded portions 488b of the projections 488 provide such a gripping point for the caregiver. Additionally, for the projections 388, 488 that extend below the lower lateral edge 376, 476 of the pocket 364, 464, such as in FIGS. 8 and 9A, the portions of the projections 388, 488 extending below the lower lateral edge 376, 476 can provide additional ease of access for gripping the projection 388, 488. As an additional benefit, the projections 388, 488 can also provide a visual cue to the caregiver that a pocket 364, 464 is present on the absorbent article 10, where otherwise it may be less noticeable.

It is to be noted that the projections 388, 488 of pockets 364, 464 of FIGS. 8-10 can be employed as an additional feature to any one of the embodiments depicted in FIGS. 1-7, or as an alternative to the embodiments depicted in FIGS. 1-7 to provide improved accessibility to the pocket 364, 464. Additionally, it is contemplated that the material 366, 466 forming the pockets 364, 464 of FIGS. 8-10 can be a portion of the outer cover 26, such as by forming a slit in an outer layer 48 of the outer cover 26 as discussed above. In such embodiments, the projection 388 could be integral to the the outer cover 26 or could be a projection 488 coupled to the outer cover 26.

Absorbent Body:

The absorbent body 34 can be suitably constructed to be generally compressible, conformable, pliable, non-irritating to the wearer's skin and capable of absorbing and retaining liquid body exudates. The absorbent body 34 can be manufactured in a wide variety of sizes and shapes (for example, rectangular, trapezoidal, T-shape, I-shape, hourglass shape, etc.) and from a wide variety of materials. For example, FIG. 1 illustrates an absorbent body 34 that is rectangular in shape. The size and the absorbent capacity of the absorbent body 34 should be compatible with the size of the intended wearer (infants to adults) and the liquid loading imparted by the intended use of the absorbent article 10. The absorbent body 34 can have a length and width that can be less than or equal to the length and width of the absorbent article 10.

The absorbent body 34 can also be further configured to help the caregiver maintain control of the pocket 64 of the absorbent article 10. For example, the absorbent body 34 and the pocket 64 can be disposed such that first end edge 35 of the absorbent body 34 can be closer to the lateral axis 31 than is the upper lateral edge 74 of the pocket 64. The space formed between the first end edge 35 of the absorbent body 34 and the upper lateral edge 74 of the pocket 64 can provide space for a caregiver's finger tips to rest when the caregiver is using the pocket 64 to wipe the wearer of the article 10.

In an embodiment, the absorbent body 34 can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent body 34 can be a matrix of cellulosic fluff and superabsorbent material. In an embodiment, the absorbent body 34 may be constructed of a single layer of materials, or in the alternative, may be constructed of two or more layers of materials.

Various types of wettable, hydrophilic fibers can be used in the absorbent body 34. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. Suitable superabsorbent materials can be selected from natural, synthetic, and modified natural polymers and materials. The superabsorbent materials can be inorganic materials, such as silica gels, or organic compounds, such as cross-linked polymers. In an embodiment, the absorbent body 34 can be free of superabsorbent material.

The absorbent body 34 can be superposed over the inner layer 50 of the outer cover 26 and can be bonded to the inner layer 50 of the outer cover 26, such as by being bonded thereto with adhesive. However, it is to be understood that the absorbent body 34 may be in contact with, and not bonded with, the outer cover 26 and remain within the scope of this disclosure. In an embodiment, the outer cover 26 can be composed of a single layer and the absorbent body 34 can be in contact with the singer layer of the outer cover 26. In an embodiment, a layer, such as but not limited to, a fluid transfer layer (not shown), can be positioned between the absorbent body 34 and the outer cover 26.

Bodyside Liner:

The bodyside liner 28 of the absorbent article 10 can overlay the absorbent body 34 and the outer cover 26 and can isolate the wearer's skin from liquid waste retained by the absorbent body 34. In various embodiments, a fluid transfer layer (not shown) can be positioned between the bodyside liner 28 and the absorbent body 34. In various embodiments, an acquisition layer (not shown) can be positioned between the bodyside liner 28 and the absorbent body 34 or a fluid transfer layer, if present. In various embodiments, the bodyside liner 28 can be bonded to the acquisition layer, or to the fluid transfer layer if no acquisition layer is present, via adhesive and/or by a point fusion bonding. The point fusion bonding may be selected from ultrasonic, thermal, pressure bonding, and combinations thereof.

In an embodiment, the bodyside liner 28 can extend beyond the absorbent body 34 and/or a fluid transfer layer, if present, and/or an acquisition layer, if present, to overlay a portion of the outer cover 26 and can be bonded thereto by any method deemed suitable, such as, for example, by being bonded thereto by adhesive, to substantially enclose the absorbent body 34 between the outer cover 26 and the bodyside liner 28. The bodyside liner 28 may be narrower than the outer cover 26, but it is to be understood that the bodyside liner 28 and the outer cover 26 may be of the same dimensions, or that the bodyside liner 28 may be of greater width than the outer cover 26. It is also contemplated that the bodyside liner 28 may not extend beyond the absorbent body 34 and/or may not be secured to the outer cover 26. It is further contemplated that the bodyside liner 28 may be composed of more than one segment of material. The bodyside liner 28 can be of different shapes, including rectangular, hourglass, or any other shape. The bodyside liner 28 can be suitably compliant, soft feeling, and non-irritating to the wearer's skin and can be the same as or less hydrophilic than the absorbent body 34 to permit body exudates to readily penetrate through to the absorbent body 34 and provide a relatively dry surface to the wearer.

The bodyside liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 28. The bodyside liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, can include more than one layer of fabrics, films, and/or webs, as well as combinations thereof. For example, the bodyside liner 28 can include a support layer and a projection layer that can be hydroentagled. The projection layer can include hollow projections, such as those disclosed in U.S. Patent Application Publication No. 2014/0121623 invented by Kirby, Scott S. C. et al. In a preferred embodiment, the bodyside liner 28 includes a bodyfacing surface that provides an uneven surface at least in the front waist region 12, such as a bodyfacing surface that includes projections as disclosed in U.S. Patent Application Publication No. 2014/0121623 noted above. Such a bodyside liner 28 provides additional benefits in softness and assists in cleaning the wearer's skin when the caregiver uses the pocket 64 of the absorbent article 10 to wipe the wearer.

For example, the bodyside liner 28 can be composed of a meltblown or spunbond web of polyolefin fibers. Alternatively, the bodyside liner 28 can be a bonded-carded web composed of natural and/or synthetic fibers. The bodyside liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 28 or it can be selectively applied to particular sections of the bodyside liner 28.

In an embodiment, a bodyside liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a bodyside liner 28 can be a spunbond substrate with a basis weight from about 10 or 12 to about 15 or 20 gsm. In an embodiment, a bodyside liner 28 can be a 12 gsm spunbond-meltblown-spunbond substrate having 10% meltblown content applied between the two spunbond layers.

Although the outer cover 26 and bodyside liner 28 can include elastomeric materials, it is contemplated that the outer cover 26 and the bodyside liner 28 can be composed of materials which are generally non-elastomeric. In an embodiment, the bodyside liner 28 can be stretchable, and more suitably elastic. In an embodiment, the bodyside liner 28 can be suitably stretchable and more suitably elastic in at least the lateral or circumferential direction of the absorbent article 10. In other aspects, the bodyside liner 28 can be stretchable, and more suitably elastic, in both the lateral and the longitudinal directions 32, 30, respectively.

Leg Elastics:

Leg elastic members 44, 46 (labeled in FIG. 1) can be secured to the outer cover 26, such as by being bonded thereto by laminate adhesive, generally laterally inward of the longitudinal side edges, 18 and 20, of the absorbent article 10. The leg elastic members 44, 46 can form elasticized leg cuffs, 52 and 54, respectively, that further help to contain body exudates. In an embodiment, the leg elastic members 44, 46 may be disposed between the inner layer 50 and outer layer 48 of the outer cover 26 as shown in the cut-out region of FIG. 1 or between other layers of the absorbent article 10. The leg elastic members 44, 46 can be a single elastic member, or each leg elastic member 44, 46 can include more than one elastic member, such as illustrated herein. A wide variety of elastic materials may be used for the leg elastic members 44, 46. Suitable elastic materials can include sheets, strands or ribbons of natural rubber, synthetic rubber, or thermoplastic elastomeric materials. The elastic materials can be stretched and secured to a substrate, secured to a gathered substrate, or secured to a substrate and then elasticized or shrunk, for example, with the application of heat, such that the elastic retractive forces are imparted to the substrate. Of course, the leg elastic members 44, 46 can be omitted from the absorbent article 10 without departing from the scope of this disclosure.

Fastening System:

In an embodiment, the absorbent article 10 can include a fastener system. The fastener system can include one or more back fasteners 56 and one or more front fasteners 58, with only one front fastener 58 being shown in FIGS. 1-10. Portions of the fastener system may be included in the front waist region 12, rear waist region 14, or both. The front fastener(s) 58 can be the same material as the material 66, 166, 266, 366, 466 forming the pocket 64, 164, 264, 364, 464, as shown in FIGS. 1-10 however, the front fastener(s) 58 can be formed from a different material than the pocket material 66, 166, 266, 366, 466.

The fastener system can be configured to secure the absorbent article 10 about the waist of the wearer and maintain the absorbent article 10 in place during use in fastened configuration, an example of which is shown in FIG. 3. In an embodiment, the back fasteners 56 can include one or more materials bonded together to form a composite ear as is known in the art, such as a fastener component 60, a stretch component 61, and a hook base 62 (labeled in FIG. 1). In a preferred embodiment, the fastening component 60 on the back ears 56 is a hook material and the front fastener 58 is comprised of loop material.

Waist Elastic Members:

In an embodiment, the absorbent article 10 can have one or more waist elastic members, such as rear waist elastic member 42, which can be formed of any suitable elastic material. The rear waist elastic member 42 can be in a rear waist region 14 of the absorbent article 10. Suitable elastic materials for waist elastic members can include, but are not limited to, sheets, strands or ribbons of natural rubber, synthetic rubber, or thermoplastic elastomeric polymers. The elastic materials can be stretched and bonded to a substrate, bonded to a gathered substrate, or bonded to a substrate and then elasticized or shrunk, for example, with the application of heat, such that elastic retractive forces are imparted to the substrate. It is to be understood, however, that the waist elastic member 42 can be omitted from the absorbent article 10 without departing from the scope of this disclosure.

### EMBODIMENTS

### Embodiment 1

An absorbent article including a front waist region including a front waist edge, a rear waist region including a rear waist edge, and a crotch region, the absorbent article further including a longitudinal axis and a lateral axis defining a horizontal plane when the absorbent article is in a stretched, laid flat configuration, the absorbent article comprising: an absorbent assembly including a bodyside liner, an outer cover, and an absorbent body disposed between the bodyside liner and the outer cover, the outer cover including a body facing surface and a garment facing surface; and a pocket disposed in the front waist region, the pocket including a pocket longitudinal axis, a first side edge, a second side edge opposite from the first side edge, an upper lateral edge, a lower lateral edge, and a bottom surface, the pocket being closed with respect to the absorbent assembly at the first side edge, the second side edge, and the upper lateral edge, the pocket being open with respect to the bottom surface at the lower lateral edge such that when the absorbent article is in the stretched, laid flat configuration, a vertical gap is provided between the lower lateral edge and the bottom surface of the pocket at the pocket longitudinal axis, the vertical gap providing access to the pocket.

### Embodiment 2

The absorbent article of embodiment 1, wherein the pocket is formed from a material, the material being gathered prior to attaching the material to the absorbent assembly to provide the vertical gap.

### Embodiment 3

An absorbent article including a front waist region including a front waist edge, a rear waist region including a rear waist edge, and a crotch region, the absorbent article further including a longitudinal axis and a lateral axis defining a horizontal plane when the absorbent article is in a stretched, laid flat configuration, the absorbent article comprising: an absorbent assembly including a bodyside liner, an outer cover, and an absorbent body disposed between the bodyside liner and the outer cover, the outer cover including a body facing surface and a garment facing surface; and a pocket disposed in the front waist region, the pocket including a pocket longitudinal axis, a first side edge, a second side edge opposite from the first side edge, an upper lateral edge, and a lower lateral edge, the pocket being closed with respect to the absorbent assembly at the first side edge, the second side edge, and the upper lateral edge, the pocket being open with respect to the outer cover at the lower lateral edge, wherein a portion of the outer cover forms a bottom surface of the pocket, the bottom surface of the pocket being elastic near the lower lateral edge of the pocket such that when the absorbent article changes from the stretched, laid flat configuration to a relaxed configuration, the bottom surface of the pocket elastically retracts to provide a separation between the lower lateral edge and the bottom surface of the pocket at the longitudinal axis of the pocket, the separation providing access to the pocket.

### Embodiment 4

The absorbent article of embodiment 3, wherein an elastic material is coupled to the portion of the outer cover forming the bottom surface of the pocket.

### Embodiment 5

The absorbent article of embodiment 4, wherein the outer cover includes an outer layer and an inner layer, and wherein the elastic material is disposed between the outer layer and the inner layer.

### Embodiment 6

The absorbent article of embodiment 3, wherein when the absorbent article is in the stretched, laid flat configuration a vertical gap is present between the lower lateral edge of the pocket and the bottom surface of the pocket at the longitudinal axis of the pocket, the vertical gap providing access to the pocket.

### Embodiment 7

The absorbent article of embodiment 1, further comprising a fastening system, the fastening system including at least one back fastener in the rear waist region and at least one front fastener in the front waist region, the fastening system configured to provide a fastened configuration of the absorbent article when the fastening system is engaged and an unfastened configuration of the absorbent article when the fastening system is not engaged, wherein the vertical gap is provided when the absorbent article changes from the fastened configuration to the unfastened configuration after the fastening system is disengaged.

### Embodiment 8

The absorbent article of embodiment 1 or embodiment 2, further comprising a fastening system, the fastening system including at least one back fastener in the rear waist region and at least one front fastener in the front waist region, the fastening system configured to provide a fastened configuration of the absorbent article when the fastening system is engaged and an unfastened configuration of the absorbent article when the fastening system is not engaged, wherein the vertical gap is present prior to the fastening system being engaged in a fastened configuration for a first time, and wherein the vertical gap increases when the absorbent article changes from the fastened configuration to the unfastened configuration after the fastening system is disengaged for a first time.

### Embodiment 9

The absorbent article of embodiment 3, further comprising a fastening system, the fastening system including at least one back fastener in the rear waist region and at least one front fastener in the front waist region, the fastening system configured to provide a fastened configuration of the absorbent article when the fastening system is engaged and an unfastened configuration of the absorbent article when the fastening system is not engaged, wherein a vertical gap is provided between the lower lateral edge and the bottom surface of the pocket at the pocket longitudinal axis upon the absorbent article changing from the fastened configuration to the unfastened configuration after the fastening system is disengaged for a first time, the vertical gap providing access to the pocket when the absorbent article is in the stretched, laid flat configuration.

### Embodiment 10

The absorbent article of any one of embodiments 7-9, wherein the pocket is formed from a material, the material being extensible and not elastic.

### Embodiment 11

The absorbent article of any one of embodiments 7-10, wherein the pocket is formed from a material, the material being in a pleated configuration.

### Embodiment 12

An absorbent article including a front waist region including a front waist edge, a rear waist region including a rear waist edge, and a crotch region, the absorbent article further including a longitudinal axis and a lateral axis defining a horizontal plane when the absorbent article is in a stretched, laid flat configuration, the absorbent article comprising: an absorbent assembly including a bodyside liner, an outer cover, and an absorbent body disposed between the bodyside liner and the outer cover, the outer cover including a body facing surface and a garment facing surface; and a pocket disposed in the front waist region and being formed from a material, the pocket including a first side edge, a second side edge opposite from the first side edge, an upper lateral edge, and a lower lateral edge, the pocket being closed with respect to the absorbent assembly at least at the first side edge and the second side edge, the pocket being open with respect to the absorbent assembly at the lower lateral edge, the pocket including a projection in a central region of the pocket near the lower lateral edge, the projection either being coupled to the material forming the pocket or being integral to the material forming the pocket, the projection providing access to the pocket.

### Embodiment 13

An absorbent article including a front waist region including a front waist edge, a rear waist region including a rear waist edge, and a crotch region, the absorbent article further including a longitudinal axis and a lateral axis defining a horizontal plane when the absorbent article is in a stretched, laid flat configuration, the absorbent article comprising: an absorbent assembly including a bodyside liner, an outer cover, and an absorbent body disposed between the bodyside liner and the outer cover, the outer cover including a body facing surface and a garment facing surface; and a pocket disposed in the front waist region, the pocket being formed from a material, the pocket including a first side edge, a second side edge opposite from the first side edge, an upper lateral edge, and a lower lateral edge, the pocket being closed with respect to the absorbent assembly at least at the first side edge and the second side edge, the pocket being open with respect to the absorbent assembly at the lower lateral edge, the pocket including a projection coupled to the material forming the pocket, the projection providing access to the pocket.

### Embodiment 14

The absorbent article of embodiment 12, wherein the projection is integral to the material forming the pocket.

### Embodiment 15

The absorbent article of embodiment 12 or embodiment 14, wherein the upper lateral edge includes a notch, the notch having a shape that nests with a shape of the projection.

### Embodiment 16

The absorbent article of embodiment 12, wherein the projection is coupled to the material forming the pocket.

### Embodiment 17

The absorbent article of embodiment 13 or embodiment 16, wherein the projection extends from the upper lateral edge to at least the lower lateral edge of the pocket.

### Embodiment 18

The absorbent article of embodiment 13 or embodiment 17, wherein the projection includes a bonded portion attached to the material forming the pocket and a non-bonded portion not attached to the material forming the pocket.

### Embodiment 19

The absorbent article of embodiment 18, wherein the non-bonded portion is disposed between the upper lateral edge and the lower lateral edge of the pocket.

### Embodiment 20

The absorbent article of embodiment 18, wherein at least a portion of the non-bonded portion is disposed between the lower lateral edge of the pocket and the lateral axis of the absorbent article.

### Embodiment 21

The absorbent article of embodiment 13 or embodiment 18, wherein the projection includes a bottom edge, the projection configured such that the bottom edge is closer to the lateral axis than is the lower lateral edge of the pocket.

### Embodiment 22

The absorbent article of any one of embodiment 12-18, wherein the projection includes a bottom edge, the bottom edge being substantially aligned with the lower lateral edge of the pocket.

### Embodiment 23

The absorbent article of any one of embodiments 13-22, wherein the projection is disposed in a central region of the pocket.

### Embodiment 24

The absorbent article of any one of embodiments 12-23, wherein the pocket is closed with respect to the absorbent assembly at the upper lateral edge.

### Embodiment 25

The absorbent article of any one of embodiments 1 or 3-6, further comprising a fastening system, the fastening system including at least one back fastener in the rear waist region and at least one front fastener in the front waist region, wherein the pocket is formed from a material, the material forming the front fastener.

### Embodiment 26

The absorbent article of any one of embodiments 2 or 12-24, wherein the material forming the pocket also forms a front fastener.

### Embodiment 27

The absorbent article of any one of embodiments 1 or 3-9, wherein the pocket is formed from a material, the material forming a portion of the outer cover.

### Embodiment 28

The absorbent article of any one of embodiments 2 or 10-26, wherein the material forming the pocket forms a portion of the outer cover.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An absorbent article including a front waist region including a front waist edge, a rear waist region including a rear waist edge, and a crotch region, the absorbent article further including a longitudinal axis and a lateral axis defining a horizontal plane when the absorbent article is in a stretched, laid flat configuration, the absorbent article comprising:
an absorbent assembly including a bodyside liner, an outer cover, and an absorbent body disposed between the bodyside liner and the outer cover, the outer cover including a body facing surface and a garment facing surface; and
a pocket disposed in the front waist region, the pocket including a pocket longitudinal axis, a first side edge, a second side edge opposite from the first side edge, an upper lateral edge, a lower lateral edge, and a bottom surface, the pocket being closed with respect to the absorbent assembly at the first side edge, the second side edge, and the upper lateral edge, the pocket being open with respect to the bottom surface at the lower lateral edge such that when the absorbent article is in the stretched, laid flat configuration, a vertical gap is provided between the lower lateral edge and the bottom surface of the pocket at the pocket longitudinal axis, the vertical gap providing access to the pocket.

2. The absorbent article of claim 1, wherein the pocket is formed from a material, the material being gathered prior to attaching the material to the absorbent assembly to provide the vertical gap.

3. An absorbent article including a front waist region including a front waist edge, a rear waist region including a rear waist edge, and a crotch region, the absorbent article further including a longitudinal axis and a lateral axis defining a horizontal plane when the absorbent article is in a stretched, laid flat configuration, the absorbent article comprising:
an absorbent assembly including a bodyside liner, an outer cover, and an absorbent body disposed between the bodyside liner and the outer cover, the outer cover including a body facing surface and a garment facing surface; and
a pocket disposed in the front waist region, the pocket including a pocket longitudinal axis, a first side edge, a second side edge opposite from the first side edge, an upper lateral edge, and a lower lateral edge, the pocket being closed with respect to the absorbent assembly at the first side edge, the second side edge, and the upper lateral edge, the pocket being open with respect to the outer cover at the lower lateral edge, wherein a portion of the outer cover forms a bottom surface of the pocket, the bottom surface of the pocket being elastic near the lower lateral edge of the pocket such that when the absorbent article changes from the stretched, laid flat configuration to a relaxed configuration, the bottom surface of the pocket elastically retracts to provide a separation between the lower lateral edge and the bottom surface of the pocket at the longitudinal axis of the pocket, the separation providing access to the pocket.

4. The absorbent article of claim 3, wherein an elastic material is coupled to the portion of the outer cover forming the bottom surface of the pocket.

5. The absorbent article of claim 4, wherein the outer cover includes an outer layer and an inner layer, and wherein the elastic material is disposed between the outer layer and the inner layer.

6. The absorbent article of claim 3, wherein when the absorbent article is in the stretched, laid flat configuration a vertical gap is present between the lower lateral edge of the pocket and the bottom surface of the pocket at the longitudinal axis of the pocket, the vertical gap providing access to the pocket.

7. The absorbent article of claim 1, further comprising a fastening system, the fastening system including at least one back fastener in the rear waist region and at least one front fastener in the front waist region, the fastening system configured to provide a fastened configuration of the absorbent article when the fastening system is engaged and an unfastened configuration of the absorbent article when the fastening system is not engaged, wherein the vertical gap is provided when the absorbent article changes from the fastened configuration to the unfastened configuration after the fastening system is disengaged.

8. The absorbent article of claim 1, further comprising a fastening system, the fastening system including at least one back fastener in the rear waist region and at least one front fastener in the front waist region, the fastening system configured to provide a fastened configuration of the absorbent article when the fastening system is engaged and an unfastened configuration of the absorbent article when the fastening system is not engaged, wherein the vertical gap is present prior to the fastening system being engaged in a fastened configuration for a first time, and wherein the vertical gap increases when the absorbent article changes from the fastened configuration to the unfastened configuration after the fastening system is disengaged for a first time.

9. The absorbent article of claim 3, further comprising a fastening system, the fastening system including at least one back fastener in the rear waist region and at least one front fastener in the front waist region, the fastening system configured to provide a fastened configuration of the absorbent article when the fastening system is engaged and an unfastened configuration of the absorbent article when the fastening system is not engaged, wherein a vertical gap is provided between the lower lateral edge and the bottom surface of the pocket at the pocket longitudinal axis upon the absorbent article changing from the fastened configuration to the unfastened configuration after the fastening system is disengaged for a first time, the vertical gap providing access to the pocket when the absorbent article is in the stretched, laid flat configuration.

10. The absorbent article of claim 7, wherein the pocket is formed from a material, the material being extensible and not elastic.

11. The absorbent article of claim 7, wherein the pocket is formed from a material, the material being in a pleated configuration.

12. An absorbent article including a front waist region including a front waist edge, a rear waist region including a rear waist edge, and a crotch region, the absorbent article further including a longitudinal axis and a lateral axis defining a horizontal plane when the absorbent article is in a stretched, laid flat configuration, the absorbent article comprising:
an absorbent assembly including a bodyside liner, an outer cover, and an absorbent body disposed between the bodyside liner and the outer cover, the outer cover including a body facing surface and a garment facing surface; and
a pocket disposed in the front waist region and being formed from a material, the pocket including a first side edge, a second side edge opposite from the first side edge, an upper lateral edge, and a lower lateral edge, the pocket being closed with respect to the absorbent assembly at least at the first side edge and the second side edge, the pocket being open with respect to the absorbent assembly at the lower lateral edge, the pocket including a projection in a central region of the pocket near the lower lateral edge, the projection either being coupled to the material forming the pocket or being integral to the material forming the pocket, the projection providing access to the pocket.

13. An absorbent article including a front waist region including a front waist edge, a rear waist region including a rear waist edge, and a crotch region, the absorbent article further including a longitudinal axis and a lateral axis defining a horizontal plane when the absorbent article is in a stretched, laid flat configuration, the absorbent article comprising:
an absorbent assembly including a bodyside liner, an outer cover, and an absorbent body disposed between the bodyside liner and the outer cover, the outer cover including a body facing surface and a garment facing surface; and
a pocket disposed in the front waist region, the pocket being formed from a material, the pocket including a first side edge, a second side edge opposite from the first side edge, an upper lateral edge, and a lower lateral edge, the pocket being closed with respect to the absorbent assembly at least at the first side edge and the second side edge, the pocket being open with respect to the absorbent assembly at the lower lateral edge, the pocket including a projection coupled to the material forming the pocket, the projection providing access to the pocket.

14. The absorbent article of claim 12, wherein the projection is integral to the material forming the pocket.

15. The absorbent article of claim 12, wherein the upper lateral edge includes a notch, the notch having a shape that nests with a shape of the projection.

16. The absorbent article of claim 12, wherein the projection is coupled to the material forming the pocket.

17. The absorbent article of claim 13, wherein the projection extends from the upper lateral edge to at least the lower lateral edge of the pocket.

18. The absorbent article of claim 13, wherein the projection includes a bonded portion attached to the material forming the pocket and a non-bonded portion not attached to the material forming the pocket.

19. The absorbent article of claim 18, wherein the non-bonded portion is disposed between the upper lateral edge and the lower lateral edge of the pocket.

20. The absorbent article of claim 18, wherein at least a portion of the non-bonded portion is disposed between the lower lateral edge of the pocket and the lateral axis of the absorbent article.

21. The absorbent article of claim 13, wherein the projection includes a bottom edge, the projection configured such that the bottom edge is closer to the lateral axis than is the lower lateral edge of the pocket.

22. The absorbent article of claim 12, wherein the projection includes a bottom edge, the bottom edge being substantially aligned with the lower lateral edge of the pocket.

23. The absorbent article of claim 13, wherein the projection is disposed in a central region of the pocket.

24. The absorbent article of claim 12, wherein the pocket is closed with respect to the absorbent assembly at the upper lateral edge.

25. The absorbent article of claim 1, further comprising a fastening system, the fastening system including at least one back fastener in the rear waist region and at least one front fastener in the front waist region, wherein the pocket is formed from a material, the material forming the front fastener.
